(19) United States
(12) Patent Application Publication
(10) Pub. No.: US 2004/0122058 A1
(43) Pub. Date: Jun. 24, 2004

(54) Use of specific compounds particularly kinase inhibitors for treating viral infections

(76) Inventors: Dorian Bevec, Munich (DE); Ute Stroher, Winnipeg (CA); Hans-Dieter Klenk, Linden (DE); Christian Wallasch, Munich (DE)

Correspondence Address: Leon R Yankwich, Yankwich & Associates, 201 Broadway, Cambridge, MA, 02139, US

(21) Appl. No.: 10/469,904

(22) PCT Filed: Mar. 6, 2002
(86) PCT No.: PCT/EP02/02467

(30) Foreign Application Priority Data
Mar. 6, 2001 (EP) 01105552.2

(51) Int. Cl. A61K 31/4439; A61K 31/353; A61K 31/277; A61K 31/165

(52) U.S. Cl. 514/341; 514/456; 514/523; 514/617

(57) ABSTRACT

The present invention relates to the use of specific compounds for the treatment and/or prophylaxis of inflammatory conditions. Furthermore, the present invention relates to the use of inhibitors for treating viral infections, particularly to the use of MEK inhibitors, especially MEKI inhibitors, for prophylaxis and/or treatment of virally induced hemorrhagic fever and/or hemorrhagic shock syndromes, for the treatment of virally induced TNF-∝ mediated diseases, and for regulating and/or inhibiting of virally induced TNF-∝ production. Furthermore, methods for preventing and/or treating of virally induced hemonrhagic fevers and/or hemorrhagic shock syndromes, for regulating and/or inhibiting virally induced TNF-∝ production, and for the treatment of virally induced TNF-÷ medicated diseases are disclosed together with pharmaccutical compositions useful within said methods.

### SPECIFICATION

The present invention relates to the use of specific compounds for the treatment and/or prophylaxis of inflammatory conditions. Moreover, the present invention relates to the use of inhibitors (small molecular weight compounds) for treating viral infections. In addition, the present invention relates to the use of inhibitors (small molecular weight compounds) of the protein kinases MEK, especially inhibitors of the protein kinase MEK1, for prophylaxis and/or treatment of virally induced hemorrhagic fever and/or hemorrhagic shock syndromes, for the treatment of virally induced TNF-α mediated diseases, and for regulating and/or inhibiting of virally induced TNF-α production. Furthermore, methods for preventing and/or treating of virally induced hemorrhagic fevers and/or hemorrhagic shock syndromes, for regulating and/or inhibiting virally induced TNF-α production, and for the treatment of virally induced TNF-A mediated diseases are disclosed together with pharmaceutical compositions useful within said methods.

### INTRODUCTION

Cytokines are soluble glycoproteins (proteins coated with sugars) released by cells of the immune system. The immune system is a collection of cells (such as B-Cells, T-Cells, etc.), chemical messengers (e.g. cytokine) and proteins (such as immunoglobulin) that work together to protect the body from potentially harmful, infectious microorganisms, such as bacteria, viruses and fungi.

B-Cells are white blood cells which develop from B stem cells into plasma cells which produce immunoglobulins (antibodies). The immune system plays a role in the control of cancer and other diseases, but is also the culprit in the phenomena of allergies, hypersensitivity and the rejection of transplanted organs, tissues and medical implants.

Cytokines act nonenzymatically through specific receptors to regulate immune responses. Cytokines resemble hormones in that they act at low concentrations bound with high affinity to a specific receptor.

Tumor necrosis factors are found in two forms:

### 1. TNF-α

### 2. TNF-β

The genes that encode both TNF forms are found in the major histocompatibility complex (MHC). MHC is a cluster of genes on chromosome 6 in humans, encoding cell surface molecules that are polymorphic and that code for antigens which lead to rapid graft rejection between members of a single species which differ at these loci. Several classes of protein such as MHC class I and II proteins are encoded in this region. MHC class I molecule is a molecule encoded to genes of the MHC which participates in antigen presentation to cytotoxic T (CD8+) cells. MHC class II molecule is a molecule encoded by genes of the MHC which participates in antigen presentation to helper T (CD4+) cells. These in humans, are known as human leukocyte antigens (HLA). Class I molecules are designated HLA-A, B, or C. Class II molecules are designated DP, DQ or DR.

Human leukocyte antigens (HLA) are proteins located on the surface of white blood cells which play an important role in our body's immune response to foreign substances. TNF-α is produced predominantly by macrophages and some other cells. Macrophages are large white blood cells, derived from monocytes (a subclass of mononuclear leukocytes). Monocytes are white blood cells which can ingest dead or damaged cells (through phagocytosis) and provide immunological defenses against many infectious organisms. Monocytes migrate into tissues and develop into macrophages.

T-Cells or T-lymphocytes (white blood cells) that develop in the bone marrow, matures in the thymus and express what appear to be antibody molecules on their surfaces but, unlike B-cells, these molecules cannot be secreted. This is called a T-Cell Receptor (CD3, and CD4 or CD8) and works as part of the immune system in the body and produces cytokine in order to help B-Lymphocytes to produce immunoglobulins.

Cytokines like ILl and TNF act alone or together to induce systemic inflammation (e.g., fever). LPS (Lipopolysaccharide), an endotoxin from bacterial cell wall, stimulates production of TNF-α. TNF is also chemotactic for neutrophils and monocytes. Neutrophils are leukocytes (white blood cells) of the polymorphonuclear leukocyte subgroup. Neutrophils form a primary defense against bacterial infections.

TNF is also thought to upregulate HIV replication, and may contribute to the pathogenesis of wasting (cachexia) due to the loss of fat from fat cells and increased metabolism of muscle cells. TNF causes the symptoms associated with bacterial infections (septic shock, fever, muscle ache, lethargy, headache, nausea and inflammation).

Shock means a life-threatening condition where blood pressure is too low to sustain life. A shock occurs when a low blood volume (due to severe bleeding, excessive fluid loss or inadequate fluid uptake), inadequate pumping action of the heart or excessive dilation of the blood vessel walls (vasodilation: excessive relaxation or dilation of the blood vessel walls) causes low blood pressure. This in turn results in inadequate blood supply to body cells, which can quickly die or be irreversibly damaged.

Tumor Necrosis Factor Superfamily

The first suggestion that a tumor necrotizing molecule existed was made when it was observed that cancer patients occasionally showed spontaneous regression of their tumors following bacterial infections (Coley, W.B. (1981) Ann. Surg. 14:199). Subsequent studies in the 1960s indicated that host-associated (or endogenous) mediators, manufactured in response to bacterial products, were likely responsible for the observed effects (Beutler, B. & A. Cerami (1989) Annu. Rev. Immunol. 7:625; O'Malley, W.E. et al. (1962) J. Nati. Cancer Inst. 29:1169). In 1975, it was shown that a bacterially-induced circulating factor had strong anti-tumor activity against tumors implanted in the skin in mice (Carswell, E.A. et aL (1975) Proc. NatI. Acad. Sci. USA 72:3666). This factor, designated tumor necrosis factor (TNF), was subsequently isolated (Aggarwal, B.B. et aL (1985) J. Biol. Chem. 260:2345), cloned (Pennica, D. et al. (1984) Nature 312:724), and found to be the prototype of a family of molecules that are involved with immune regulation and inflammation (Gruss, H- J. & S. K. Dower (1995) Blood 85:3378; Cosman, D. (1996) In Blood Cell Biochemistry, Vol. 7:Hematopoietic Cell Growth Factors and Their Receptors, Wheften, A. D. and J. Gordon, eds., Plenum Press, New York). The receptors for TNF and the other members of the TNF superfamily also constitute a superfamily of related proteins (Baker, S. J. & E. P. Reddy (1996) Oncogene 12:1; Lotz, M. et aL. (1996) J. Leukoc. Biol. 60:1; Armitage, R. J. (1994) Curr. Opin. Immunol. 6:407; Ware, C. F. et a/. (1996) J. Cell. Biochem. 60:47).

Ligands/Co-Receptors

TNF-related ligands usually share a number of common features. These features do not include a high degree of overall amino acid (aa) sequence homology. With the exception of nerve growth factor (NGF) and TNF-β, all ligands are synthesized as type II transmembrane proteins (extracellular C-terminus) that contain a short cytoplasmic segment (10-80 aa residues) and a relatively long extracellular region (140-215 aa residues). NGF, which is structurally unrelated to TNF, is included in this superfamily only because of its ability to bind to the TNFRSF low affinity NGF receptor (LNGFR). NGF has a classic signal sequence peptide and is secreted. TNF-β, in contrast, although also fully secreted, has a primary structure much more related to type II transmembrane proteins. TNF-β might be considered as a type II protein with a non-functional, or inefficient, transmembrane segment. In general, TNFSF members form trimeric structures, and their monomers are composed of beta-strands that orient themselves into a two sheet structure. As a consequence of the trimeric structure of these molecules, it is suggested that the ligands and receptors of the TNSF and TNFRSF superfamilies undergo “clustering” during signal transduction (Cosman, D. (1994) Stem Cells 12:440).

TNF-α: Human TNF-A is a 233 aa residue, nonglycosylated polypeptide that exists as either a transmembrane or soluble protein (Shirai, T. et aL (1985) Nature 313:803; Wang, A.M. et aL (1985) Science 228:149). When expressed as a 26 kDa membrane bound protein, TNF-α consists of a 29 aa residue cytoplasmic domain, a 28 aa residue transmembrane segment, and a 176 aa residue extracellular region. The soluble protein is created by a proteolytic cleavage event via an 85 kDa TNF-A converting enzyme (TACE), which generates a 17 kDa, 157 aa residue molecule that normally circulates as a homotrimer (Kreigler, M. et al. (1988) Cell 53:45; Smith, R.A. & C. Baglioni (1987) J. Biol. Chem. 262:6951). Normal levels of circulating TNF are reported to be in the 10-80 pg/mL range (Steinshamn, S. etal. (1995) Br. J. Haematol. 89:719; Spengler, U. et aL (1996) Cytokine 8:864). While both membrane-bound and soluble TNF-α are biologically active, soluble TNF-α is reported to be more potent (Decoster, E. et a/. (1995) J. Biol. Chem. 270:18473). Mouse to human, full-length TNF- shows 79% aa sequence identity (Pennica, D. et aL (1985) Proc. Natl. Acad. Sci. USA 82:6060; Fransen, L. et al. (1985) Nucleic Acids Res. 13:4417). The variety of cell types known to express TNF-α is enormous and includes macrophages, CD4⁺and CD8⁺T cells (Ware, C. F. et al. (1992) J. Immunol. 149:3881),. adipocytes (Kem, P.A. et al. (1995) J. Clin. Invest. 95:2111), keratinocytes (Lisby, S. et al. (1995) Int. Immunol. 7:343), mammary and colon epithelium (Varela, L.M. & M.M. (1996) Endocrinology 137:4915; Jung, H.C. et al. (1995) J. Clin. Invest. 95:55), osteoblasts (Modrowski, D. et al. (1995) Cytokine 7:720), mast cells (Bissonnefte, E. Y. et al. (1995)

Immunology 86:12), dendritic cells (Zhou, L-J. & T. F. Tedder (1995) Blood 86:3295), pancreatic beta-cells (Yamada, K. et aL (1993) Diabetes 42:1026), astrocytes (Lee, S. C. et al. (1993) J. Immunol. 150:2659), neurons (Tchelingerian, J-L. et a/. (1996) J. Neurosci. Res. 43:99), monocytes (Frankenberger, M. et al. (1996) Blood 87:373), and steroid-producing cells of the adrenal zona reticularis (Gonzalez-Hemandez, J.A. etal. (1996)J. Clin. Endocrinol. Metab. 81:807).

Tumor necrosis factors alpha and beta are cytokines that bind to common receptors on the surface of target cells and exhibit several common biological activities. TNF-α also shares an important inflammatory property with IL-6 and IL-11, i.e. the induction of acute phase reactant protein production by the liver. TNF-α and IL-1 further exert secondary inflammatory effects by stimulating ILL synthesis in several cell types. IL-6 then mediates its own effects and those of TNF-α and IL-1 in inducing fever and the acute phase response, thereby perpetuating the inflammatory response through a cascade of cytokines with overlapping properties.

Although in general the effects of cytokines are exerted locally at the site of their production (autocrine and paracrine), TNF-α and TNF-β as well as IL-1 and IL-6, have major systemic (endocrine) effects when either produced acutely in large amounts, as in the case of bacterial sepsis, or chronically in lesser amounts, as in the case of chronic infections. During sepsis with Gram negative organisms, lipopolysaccharides (endotoxin) released from bacteria trigger the widespread production of TNF-α (and subsequently IL-1 and IL6) by macrophages. The systemic release of these cytokines has been shown to be responsible for the fever and hypotension that characterize septic shock.

NGF: Human NGF is a 12.5 kDa, nonglycosylated polypeptide 120 aa residues long (Ullrich, A. et aL (1983) Nature 303:821; Scott, J. et al. (1983) Nature 302:538). Synthesized as a prepropeptide, there is an 18 aa residue signal sequence, a 103 aa residue N-terminal pro-sequence, and a 120 aa residue mature segment. Human to mouse, there is 90% aa sequence identity in the mature segment. In the mouse, NGF is referred to as beta-NGF, due to the existence of NGF in a 130 kDa (7S) heterotrimeric (apy) complex in submaxillary glands. Many cells, however, do not synthesize all the components of this 7S complex, and the typical form for NGF is a 25 kDa, non-disulfide linked homodimer (Edwards, R.H. et al. (1988) J. Biol. Chem. 263:6810). NGF and all other neurotrophins bind to the LNGFR, a member of the TNFRSF (Chao, M.V. (1994) J. Neurobiol. 25:1373).

CD40L: Human CD40L is a 39 kDa, type 11 (extracellular Cterminus) transmembrane glycoprotein that was originally identified on the surface of CD4+T cells (Hollenbaugh, D. etaL (1992) EMBO J. 11:4313). Wth a predicted molecular weight of 29 kDa, CD40L is 261 aa residues long, with a 22 aa residue cytoplasmic domain, a 24 aa residue transmembrane segment, and a 215 aa residue extracellular region. Human to mouse, CD40L is 73% identical at the aa sequence level and mouse CD40L is apparently active in humans (Armitage, R.J. et al. (1992) Nature 357:80). Although usually considered to be a membrane bound protein, natural, proteolytically cleaved 15-18 kDa soluble forms of CD40L with full biological activity have also been described (Pietravalle, F. et aL. (1996) J. Biol. Chem. 271:5965; Pietravalle, F. et aL (1996) Eur. J. Immunol. 26:725). Like TNF-α , CD40L is reported to form natural trimeric structures (Pietsch, M. C. & C. V. Jongeneel (1993) Int. Immunol. 5:233). Cells known to express CD40L include B- cells, CD4+and CD8+T-cells (Desai-Mehta, A. et al. (1996) J. Clin. Invest. 97:2063), mast cells and basophils (Gauchat, J-F. et al. (1993) Nature 365:340), eosinophils (Gauchat, J-F. et aL (1995) Eur. J. Immunol. 25:863), dendritic cells (Pinchuk, L.M. et al. (1996) J. Immunol. 157:4363), and monocytes, NK cells, and y8 T-cells (Cocks, B.G. etaL. (1993) Int. Immunol. 5:657).

CD137U4-lBBL: Mouse 4-1BBL is a 50 kDa, 309 aa residue transmembrane glycoprotein that is the largest of the TNFSF members (Goodwin, R.G. et al. (1993) Eur. J. Immunol. 23:2631). With a predicted molecular weight of 34 kDa, the molecule has an 82 aa residue cytoplasmic region, a 21 aa residue transmembrane segment, and a 206 aa residue extracellular domain. Although human and mouse 4-1 BB molecules exhibit 60% identity at the aa level, human and mouse 4-1BBL molecules exhibit only 36% identity at the aa level. This level of cross species conservation is much lower than that shown by other members of the TNFSF (Alderson, M.R. et a/. (1994) Eur. J. Immunol. 24:2219). In mice, two ligands are known for 4-1BB: 4-IBBL and laminin (Loo, D.T. et al (1997) J. Biol. Chem. 272:6448). Cells known to express 4-1BBL include B-cells, dendritic cells, and macrophages (DeBenedette, M.A. etaL (1997) J. Immunol. 158:551; Pollok, K.E. et al (1994) Eur. J. Immunol. 24:367).

CD134UOX40L: OX40, the receptor for OX40L, is a T cell activation marker with limited expression that seems to promote the survival (and perhaps prolong the immune response) of CD4+T cells at sites of inflammation. OX40L also shows limited expression. Currently only activated CD4+, CD8+T cells, B-cells, and vascular endothelial cells have been reported to express this factor (Imura, A. et al. (1996) J. Exp. Med. 183:2185). The human ligand is a 32 kDa, 183 aa residue glycosylated polypeptide that consists of a 21 aa residue cytoplasmic domain, a 23 aa residue transmembrane segment, and a 139 aa residue extracellular region. When compared to the extracellular region of TNFe, OX40L has only 15% aa sequence identity, again emphasizing the importance of secondary and tertiary structures as the basis for inclusion in the TNF Superfamily. Human OX40L is 46% identical to mouse OX40L at the aa sequence level. Mouse OX40L is active in humans, but human OX40L is inactive in mice. Consistent with other TNFSF members, OX40L is reported to exist as a trimer (AlShamkhani, A. et al. (1997) J. Biol. Chem. 272:5275).

CD27UCD70: Human CD27L is a 50 kDa, 193 aa residue type II (extracellular C-terminus) transmembrane glycoprotein that appears to have a very limited immune system expression pattem (Goodwin, R.G. et al. (1993) Cell 73:447; Bowman, M.R. et al. (1994) J. Immunol. 152:1756). Having less than 25% aa sequence identity to TNF-α and CD40L, the molecule has only a 20 aa residue cytoplasmic segment, an 18 aa residue transmembrane domain, and a 155 aa residue extracellular region. Although the 20 aa residue cytoplasmic segment is short by most standards, there is a suggestion that it has a signaling function, perhaps activating the cytolytic program of γδ T-cells (Orengo, A.M. et aL (1997) Clin. Exp. Immunol. 107:608) and/or contributing necessary signals for antibody production in B cells (Lens, S.M.A. etal. (1996) Eur. J. Immunol. 26:2964). Cells known to express CD27L are usually activated cells and include NK cells (Yang, F.C. et aL (1996) Immunology 88:289), B-cells (Agematsu, K. et al. (1995) J. Immunol. 154:3627), CD45RO+, CD4+and CD8+T cells, y8 T-cells, and certain types of leukemic B cells (Ranheim, E. K. et al (1995) Blood 85:3556).

FasL: Fas ligand (FasL) is a highly conserved, 40 kDa transmembrane glycoprotein that occurs as either a membrane bound protein or a circulating homotrimer (Takahashi, T. et aL (1994) mnt. Immunol. 6:1567; Tanaka, M. et al. (1995) EMBO J. 14:1129). In humans, FasL is synthesized as a 281 aa residue protein with an 80 aa residue cytoplasmic region, a 22 aa residue transmembrane segment, and a 179 aa residue extracellular domain. When proteolytically cleaved, FasL is a 70 kDa homotrimer composed of 26 kDa monomers with full biological activity. In mice, the FasL is somewhat different. Although mouse FasL molecule has 77% aa sequence identity with human FasL (Lynch, D.H. et al. (1994) Immunity 1:131; Takahashi, T: et al. (1994) Cell 76:969), polymorphisms exist in the mouse FasL, leading to functionally distinct FasL forms (Kayagaki, N. et aL (1997) Proc. Nat]. Acad. Sci. USA 94:3914). In addition, a one aa residue substitution at position 273 (Phe to Leu) results in the gid/gid (generalized lymphoproliferative disease) mutation. Finally, while FasL in a membrane-bound form shows species cross- reactivity, soluble mouse FasL is apparently biologically inactive. Cells known to express FasL include type 11 pneumocytes and bronchial epithelium (Niehans, G.A. etal. (1997) Cancer Res. 57:1007), monocytes (Oyaizu, N. et aL (1997) J. Immunol. 158:2456), LAK cells and NK cells (Lee, R.K. et al. (1996) J. Immunol. 157:1919; Arase, H. et al. (1995) J. Exp. Med. 181:1235), dendritic cells (Lu, L. et al (1997) J. Immunol. 158:5676), B-cells (Hahne, M. et aL (1996) Eur. J. Immunol. 26:721), macrophages (Badley, A.D. et al. (1996) J. Virol. 70:199), CD4+and CD8+T cells (Hanabuchi, S. et aL (1994) Proc. Nati. Acad. Sci. USA 91:4930), and colon and lung carcinoma cells (Shiraki, K. et aL (1997) Proc. Natl. Acad. Sci. USA 94:6420).

CD30L: Human CD30L is a 40 kDa, 234 aa residue transmembrane glycoprotein with 72% aa sequence identity to its mouse counterpart (Smith, C.A. et al. (1993) Cell 73:1349). With a predicted molecular weight of 26 kDa, the molecule consists of a 46 aa residue cytoplasmic region, a 21 aa residue transmembrane segment, and a 172 aa residue extracellular domain. Species cross-reactivity has been reported. As suggested for CD27L, the cytoplasmic region is suggested to transduce a signal (Wiley, S.R. et al. (1996) J. Immunol. 157:3635). The CD30/CD30L system is complex since CD30 ligation can induce both proliferation and apoptosis. Cells known to express CD30L include monocytes and macrophages, B cells plus activated CD4+and CD8+T cells (Younes, A. et al. (1996) Br. J. Haematol. 93:569), neutrophils, megakaryocytes, resting CD2+T cells, erythroid precursors (Gaftei, V. et aL (1997) Blood 89:2048), and eosinophils (Pinto, A. et al. (1996) Blood 88:3299).

TNF-β/LT-α: TNF-β, otherwise known as lymphotoxin-alpha (LT-alpha) is a molecule whose cloning was contemporary with that of TNF-α (Gray, P.W. et aL (1984) Nature 312:721). Although TNF-β, circulates as a 171 aa residue, 25 kDa glycosylated polypeptide, a larger form has been found that is 194 aa residues long (Aggarwal, B.B. et al. (1985) J. Biol. Chem. 260:2334). The human TNF-β cDNA codes for an open reading frame of 205 aa residues (202 in the mouse) (Gardner, S. M. et al. (1987) J. Immunol. 139:476), and presumably some type of proteolytic processing occurs during secretion. As with TNF-α , circulating TNF-β, exists as a non-covalently linked trimer and is known to bind to the same receptors as TNF-α (Hochman, P. S. et al. (1996) J. lnflamm. 46:220; Li, C-B. et aL (1987) J. Immunol. 138:4496; Browning, J. L. et al. (1996) J. Immunol. 154:33; Eck, M.J. et al. (1992) J. Biol. Chem. 267:2119). Circulating TNF-β levels are reported to be about 150 pg/mL (Sriskandan, S. et al. (1996) Cytokine 8:933). TNF-α to TNF-β, aa sequence identity is reported to be 28%. Unlike TNF-α , TNF-β does not have a transmembrane form. However, it can be membrane-associated, due to its binding to membrane-anchored LT-beta (see below) (Ware, C.F. et aL (1992) J. Immunol. 149:3881). In this complex, TNF-β and LT-beta will form a heterotrimer that binds to both the LT-beta receptor and TNFRI receptor. Activation of the TNFRI receptor, however, does not occur. Cells known to express TNF-β include NK cells, T cells and B cells. TNF-β binds to the same high affinity receptors as TNF-α . Its properties are similar to those of TNF-α and include the induction of apoptosis (programmed cell death) in many types of transformed, virally infected, and tumor cells, and the simulation of several PMN effector functions. LT-beta: Human lymphotoxin-beta (LT-beta), also known as p33, is a 33 kDa type II (extracellular C-terminus) transmembrane glycoprotein originally cloned from a T cell hybridoma cell line. It is 244 aa residues long, and has a 16 aa residue cytoplasmic segment, a 31 aa residue transmembrane domain, and a 197 aa residue extracellular region (Browning, J.L. et al. (1993) Cell 72:847). On the membrane surface, LT-beta readily forms a trimeric complex with TNF-β,1, in either a 2:1 (major form) or a 1:2 (minor form) ratio. LT-beta is not secreted. A comparison of human to mouse LT-beta shows 80% aa sequence identity in homologous regions (Lawton, P. et al. (1995) J. Immunol. 154:239). Overall, however, the mouse gene shows significant differences from the human gene. In mice, an intron has been incorporated into the genome creating a 66 aa residue insert into what would otherwise be a 240 aa residue molecule (Pokholok, D.K. et aL (1995) Proc. Natl. Acad. Sci. USA 92:674).

TRAIL: TRAIL, or TNF-related apoptosis-inducing ligand, is a newly discovered TNFSF member initially cloned from human heart and lymphocyte cDNA libraries (Wiley, S.R. et al. (1995) Immunity 3:673). With a predicted molecular weight of 32 kDa, human TRAIL is 281 aa residues long, with a 17 aa residue cytoplasmic tail, a 21 aa residue transmembrane segment, and 243 aa residue extracellular region (Pitti, R.M. etaL (1996) J. Biol. Chem. 271:12687). Human TRAIL is 65% identical to mouse TRAIL at the aa sequence level across the entire molecule and there is complete species cross-reactivity. As a membrane bound protein, TRAIL shows a trimeric structure. Although TRAIL is known to be expressed by lymphocytes, many tissues seem to express the ligand, and this broad expression paftem suggests an intriguing function for the molecule.

Receptors

As with members of the TNF Superfamily, members of the TNF Receptor Superfamily (TNFRSF) also share a number of common features. In particular, molecules in the TNFRSF are all type I (N-terminus extracellular) transmembrane glycoproteins that contain one to six ligand-binding, 40 aa residue cysteine-rich motifs in their extracellular domain. In addition, functional TNFRSF members are usually trimeric or multimeric complexes that are stabilized by intracysteine disulfide bonds. Unlike most members of the TNFSF, TNFRSF members exist in both membrane-bound and soluble forms. Finally, although aa sequence homology in the cytoplasmic domains of the superfamily members does not exceed 25%, a number of receptors are able to transduce apoptotic signals in a variety of cells, suggesting a common function (Yuan, J. (1997) Curr. Opin. Cell Biol. 9:247).

Examples of the TNF receptor superfamily are the human low-affinity nerve growth factor receptor (LNGFR), the activation-induced glycoprotein CD137/4-1 BB/ILA, the variably glycosylated polypeptide CD27 (cells known to express CD27 include NK cells, B-cells, CD4+, CD8+T-cells and thymocytes), the transmembrane protein DR4 (Death Receptor 4) which is one of three known receptors for TRAIL, the Death Receptor 5 (DR5) which is the second of three known receptors for TRAIL, the transmembrane protein GITR (glucocorticoid-induced TNFR family-related) that is suggested to be a close relative of 4-1 BB and CD27, the Osteoprotegerin/OPG and the transmembrane glycoproteins CD40 (most often associated with B cell proliferation and differentiation) (van Kooten, C. & J. Banchereau (1996) Adv. Immunol. 61:1), CD134/OX40/ACT35 (OX40 is type I external N-terminus transmembrane glycoprotein that appears to have a very limited paftem of expression, currently consisting of only activated CD4⁺and CD8⁺T cells), TNFRI/p55/CD1 20a (TNFRI is apparently expressed by virtually all nucleated mammalian cells), TNFRII/p75/CD120b, Fas/CD95/APO-1 (Human fibroblast associated (Fas) transmembrane glycoprotein is found on multiple cell types), CD30/Ki-1 (often associated with the Reed-Stemberg cells of Hodgkin's disease), LT-beta R (lymphotoxin-beta receptor), DR3IWSL-lfTRAMP/APO-3/LARD (DR3 or Death Receptor 3 has been isolated under a variety of names), DcRlTRID (DcRl or Decoy Receptor-1 or TRAIL Receptor without an Intracellular Domain (TRID) is a membrane-bound receptor for TRAIL that possesses no cytoplasmic domain), TR2 (is a newly discovered type I transmembrane glycoprotein that has no known ligand at present).

Viral Hemorrhagic Fever

The term viral hemorrhagic fever (VHF) refers to the illness associated with a number of geographically restricted viruses. This illness is characterized by fever and, in the most severe cases, shock and hemorrhage (Fisher-Hoch SP, Simpson DIH. Dangerous pathogens. Br Med Bull 1985;41: 391-5). A number of other febrile viral infections may produce hemorrhage and the agents of Lassa, Marburg, Ebola, and Crimean-Congo hemorrhagic fevers are known to have caused significant outbreaks of disease with person-to-person transmission.

The increasing volume of international travel, including visits to rural areas of the tropical world, provides opportunity for the importation of these infections into countries with no endemic VHF, such as the United States.

LASSA FEVER

Lassa virus, named after a small town in northeastern Nigeria, is an enveloped, single-stranded, bisegmented ribonucleic acid (RNA) virus classified in the family Arenaviridae. Its natural host is the multimammate rat Mastomys natalensis. This ubiquitous African rodent lives in close association with humans and is commonly found in and around houses in rural areas. The rats are infected throughout life and shed high levels of virus in their urine. Closely related viruses are found in other areas, but their potential for causing human disease is poorly understood.

Lassa fever was first recognized in 1969 in northern Nigeria. Naturally occurring 5 infections, often associated with subsequent nosocomial outbreaks, have been recognized in Nigeria, Sierra Leone, and Liberia (Monath TP. Lassa fever: review of epidemiology and epizootiology. Bull WHO 1975;52:577-92). Under natural circumstances, infection with Lassa virus occurs through contact with M. natalensis or its excreta. Currently, no vaccine is available for use in humans.

EBOLA HEMORRHAGIC FEVER

Ebola virus is a single-stranded, unsegmented, enveloped RNA virus with a characteristic filamentous structure. When magnified several thousand times by an electron microscope, these viruses have the appearance of long filaments or threads. Classification of the virus in the new family Filoviridae has been accepted. Ebola has three subtypes (Zaire, Sudan, and Reston) which have common as well as unique epitopes (Kiley MP, Bowen ETW, Eddy GA, et al. Filoviradac: a taxonomic home for Marburg and Ebola viruses? Intervirology 1982;18:2432). Ebola virus was discovered in 1976 and was named after a small river in northwest Zaire, Africa, where it was first detected. It is morphologically similar to, but antgenically distinct from Marburg virus. The reservoir of the virus in nature remains unknown.

Two epidemics occurred within a short time of each other, the first in southern 25 Sudan (World Health Organization. Ebola haemorrhagic fever in Sudan, 1976:

Report of a WHO/International Study Team. Bull WHO 1978;56:247-70) and the second in northwest Zaire (World Health Organization. Ebola haemorrhagic fever in Zaire, 1976: Report of an International Commission. Bull WHO 1978;56:271-93). The mortality rate is between about 30 to 90%.

The mode of acquiring natural infection with Ebola virus is unknown, but secondary person-to-person transmissions are described. The incubation period ranges from 2 to 21 days; the average is approximately 1 week. The illness-to-infection ratio for Ebola virus is unknown. The onset of illness is abrupt, and initial symptoms resemble those of an influenza-like syndrome.

MARBURG HEMORRHAGIC FEVER

Marburg virus is a single-stranded, unsegmented, enveloped RNA virus that is morphologically identical to, but antigenically distinct from Ebola virus. Classification of the virus in the new family Filoviridae has been accepted. Marburg virus is named after the town in Germany where some of the first cases were described (Martini GA, Siegert R, eds. Marburg virus disease. Berlin: Springer-Verlag). Its reservoir in nature remains unknown.

The mode of acquiring natural infection with Marburg virus is unknown, but secondary spread results from close contact with infected persons or contact with blood or body secretions or excretions are described. The illness-to-infection ratio is unknown but seems high for primary infections.

CRIMEAN-CONGO HEMORRHAGIC FEVER

Crimean-Congo hemorrhagic fever (CCHF) virus is an enveloped, single-stranded RNA Bunyaviridae. A hemorrhagic fever that had long been recognized in Asia came to international attention after a disease outbreak in the Crimean peninsula in 1944 and 1945 (Hoogstraal H. The epidemiology of tick-bome Crimean-Congo hemorrhagic fever in Asia, Europe, and Africa. J Med Entomol 1979;15:307417). The causative agent was later recognized to be identical to the Congo virus (Chumakov MP, Smirnova SE, Tkachenko EA. Relationship between strains of Crimean haemorrhagic fever and Congo viruses. Acta Virol 1970;14:82-5), isolated in Zaire, hence the name CCHF. Many wild and domestic animals act as reservoirs for the virus, including cattle, sheep, goats, and hares.

Nosocomial transmission is well described in recent reports from Pakistan, Iraq, Dubai, and South Africa (Swanepoel R, Shepherd AJ, Leman PA, et al. Epidemiologic and clinical features of Crimean-Congo hemorrhagic fever in southern Africa. Am J Trop Med Hyg 1978;36:120-32). The incubation period for CCHF is about 2-9 days.

DENGU and DENGUE HEMORRHAGIC FEVER

Dengue and dengue hemorrhagic fever (DHF) result from infection by any of four serotypes of dengue viruses. Transmission occurs through the bite of infected Aedes mosquitoes, principally Aedes aegypb, which is also the principal urban vector of yellow fever. Hundreds of thousands of cases of dengue and DHF are reported each year in tropical regions of the Americas, Africa, Asia and Oceania. From 1980 through 1987, 879 632 cases of dengue were reported from countries in the American region ( Pinheiro FP. Dengue in the Americas 1980-1987. Epidemiol Bull 1989;10:18). Outbreaks of the more severe form of dengue, DHF, occurred in Cuba in 1981 and in Venezuela in 1989. The majority of DHF cases, however, occur in Southeast Asia. From 1981 through 1986, 796 386 cases of DHF and 9774 deaths caused by dengue were reported from countries in Southeast Asia.

Although dengue is primarily a health problem of tropical areas, one of the earliest dengue epidemics described in the medical literature occurred in Philadelphia in 1780 ( Siler JF, Hall MW, Hitchens AP. Dengue: its history, epidemiology, clinical manifestations, immunity, and prevention. Philippine J Sci 1926;29:1-312). Massive regional efforts to control A. aegypt mosquitoes in the American region during the 1950s and 1960s resulted in the successful (although unfortunately impermanent) eradication of this species from many neighboring countries, but it was never eliminated from the southeastern United States, where it continues to thrive. Outbreaks of indigenous dengue transmission occurred in Texas in 1980 and again in 1986 (Centers for Disease Control. Imported and indigenous dengue fever: United States, 1986. MMWR 1987;33:551-4). Adding to the concern of indigenous dengue transmission is the recent establishment in the United States of another known dengue vector, Aedes albopictus, which was probably imported in shipments of tires from Asia. This species was discovered in Texas in 1985, and focal infestations as far north as Illinois have subsequently been identified. Initially DHF case fatality rates, when the disease was not treated, were as high as 50%.

Object of the present invention is to provide compounds which can be used for prophylaxis and/or treatment of hemorrhagic fevers and hemorrhagic shocks and/or inflammatory conditions, for regulating and/or inhibiting virally induced TNF-α production, or for treatment of virally induced TNF-α mediated diseases, together with methods for said treatment and pharmaceutically compositions used within said methods.

This object is solved by the disclosure of the independent claims. Further advantageous features, aspects and details of the invention are evident from the dependent claims, the description, the examples and the figure of the present application.

### DESCRIPTION OF THE INVENTION

According to one aspect, the present invention provides the use of at least one compound selected from the group consisting of 2′-amino-3′-methoxyflavone, 2-(2- chloro-4-iodophenylamino)N-cyclopropylmethoxy-3,4-difluorobenzamide, 1,4-diamino-2,3-dicyano-1,4bis(2-aminophenylthio)butadiene, and 444-fluorophenyl)2-(4-hydroxyphenyl) (4-hydroxyphenyl)5-(4-pyridyl)-1H-imidazole for the prophylaxis and/or treatment of inflammatory conditions.

According to a further aspect, the present invention refers to the use of at least one compound selected from the group consisting of 2′-amino-3′-methoxyflavone, 242-chloro 4-iodophenylaminoYN-cyclopropylmethoxy-3,4difluorobenzamide, 1,4-diamino 2,3-dicyano-1,4-bis(2-aminophenylthio) butadiene, and 4-4-fluorophenyly 2-(4-hydroxyphenyl5-(4pyridyl)-l H-imidazole for the prophylaxis and/or treatment of virally induced hemorrhagic fever and/or hemorrhagic shock syndromes.

A further aspect of the present invention is directed to the use of MEK inhibitors, especially MEK1 inhibitors, for the prophylaxis and/or treatment of virally induced hemorrhagic fever and/or hemorrhagic shock syndromes and/or inflammaotory conditions.

Suitable MEK inhibitors are listed in the PCT applications WO 98/37881 and WO 00/40237. Furthermore, suitable MEK, especially MEK1 inhibitors, are 1,4diamino- 2,3-dicyano-1,4-bis(2-aminophenylthio)butadiene, 2′-amino-3′-methoxyflavone, or 2(2-4-chlororiodophenylamino)N-cyclopropylmethoxy-3,4difluorobenzamide, as well as derivatives thereof.

Surprisingly it was found that hemorrhagic fevers and/or hemorrhagic shocks only induced by viruses can be treated with MEK inhibitors while bacterially induced hemorrhagic fevers and/or hemorrhagic shocks show no significant effect during treatment with MEK inhibitors.

Thus, another aspect of the present invention relates to a method for treating or preventing especially virally induced hemorrhagic fevers and hemorrhagic shock syndromes. Said method comprises administering to a mammal infected with a virus and in need of treatment, or administering to a mammal at the risk of developing a virally induced disease associated with hemorrhagic fever a pharmaceutically effective amount of at least one MEK inhibitor, preferably at least one MEK1 inhibitor.

Shock refers to a state in which adequate perfusion to sustain the physiologic needs of organ tissues is not present. Shock and shock-like states may be produced by many conditions, including sepsis, blood loss, impaired autoregulation, and loss of autonomic tone.

In hemorrhagic shock, blood loss occurs that exceeds the body's ability to compensate and provide adequate tissue perfusion and oxygenation. This frequently is due to trauma, but it may be caused by spontaneous hemorrhage, surgery, and a host of other causes.

Failure of compensatory mechanisms in hemorrhagic shock can lead to death. Without intervention, a classic trimodal distribution of deaths is seen in severe hemorrhagic shock. An initial peak of mortality occurs within minutes of hemorrhage due to immediate exsanguination. Another peak occurs after 1 to several hours due to progressive decompensation. A third peak occurs days to weeks later due to sepsis and organ failure.

Increased permeability of endothelial cells leads to a visceral effusions, pulmonary interstitial edema, and renal tubular dysfunction, which are a component of the shock seen in patients with filovirus infection. Said shock is presumably due to substances such as tumor necrosis factor a (TNF-α ) which may increase vascular permeability (Schniltler HJ, Mahner F, Drenckhahn D, Klenk HD, Feldmann H., Replication of Marburg virus in human endothelial cells: a possible mechanism for the development of viral hemorrhagic disease. J Clin Invest 1993;91:1301-1309). Studies using tumor necrosis factor alpha show that it is the same mediators that result in the increased endothelial permeability as well as the production of shock.

Viral hemorrhagic fevers comprise, as discussed above in detail, Ebola hemorrhagic fever, Marburg hemorrhagic fever, Lassa fever, Crimean-Congo hemorrhagic fever (CCHF), and dengue hemorrhagic fever (DHF).

Viral hemorrhagic fevers are caused by viruses from four families: filoviruses, arenaviruses, flaviviruses, and bunyaviruses. The severity of viral hemorrhagic fever can range from a relatvely mild illness to death. The usual hosts for most of these viruses are rodents or arthropods (such as ticks and mosquitoes). In some cases, the natural host for the virus is not known.

People can get Ebola hemorrhagic fever by direct contact with virus-infected blood, body fluids, organs, or semen. At present, there is no known cure or treatment. Ebola hemorrhagic fever is one of the deadliest of a group of diseases called viral hemorrhagic fevers. Ebola hemorrhagic fever has occurred in outbreaks in Central Africa. Ebola hemorrhagic fever is caused by several Ebola viruses. The source of these viruses in nature is not known. Depending on the virus, the disease can get worse until the patient becomes very ill with breathing problems, severe bleeding (hemorrhage), kidney problems, and shock.

Marburg hemorrhagic fever and Ebola hemorrhagic fever are the most interesting diseases within said group of viral hemorrhagic fevers. Marburg hemorrhagic fever is a severe type of viral hemorrhagic fever which affects both humans and non- human primates. Caused by a genetically unique zoonotic (that means, animal- bome) RNA virus of the filovirus family, its recognition led to the creation of this virus family.

Preferred is the use of the MEK inhibitors, especially MEK1 inhibitors, of the present invention for the prophylaxis and/or treatment of hemorrhagic fever and/or hemorrhagic shock syndromes induced by a filovirus, a arenavirus, a flavivirus, or a bunyavirus. Most preferred is the use of said MEK inhibitors for the prophylaxis and/or treatment of hemorrhagic fever and/or hemorrhagic shock syndromes induced by a filovirus.

In relation to the above disclosure another aspect of the present invention is directed to a method for preventing hemorrhagic fever and hemorrhagic shock syndroms induced by filoviruses.

The filovirus family comprises the Marburg virus, Ebola virus and Reston virus. Marburg virus isolates appear to belong to a single species, but there are three known subtypes of Ebola that differ significantly from each other (Ellis DS, Stamford S, Tovey DG, et al. Ebola and Marburg viruses: II. Their development within vero cells and the extra-cellular formation of branched and torus forms, J. Med. Chem. 1979, 4, 213-225). Comparison of 1172 nucleotides from the GP gene shows more than a 40% difference between any pair of the three subtypes from Sudan, Zaire, and Reston.

The Ebola virus is a bacilliform rod that contains a negative-sense RNA genome (Sanchez A, Kiley MP. Identification and analysis of Ebola virus proteins. Virology 1985;147:169). Various animals including monkeys, guinea pigs, suckling mice, and hamsters have been infected with Ebola virus. From the three subtypes of Ebola, the Zaire subtype is highly virulent and usually leads to death, the Sudan subtype often causes a self-limited infection in mice, guinea pigs, and occasionally in monkeys, while the Reston subtype is less pathogenic for monkeys and guinea pigs compared to the other subtypes.

The Marburg virus replicates in human monocytes/macrophages, resulting in cytolytic infection and release of infectious virus particles. Replication also leads to intracellular budding and accumulation of viral particles in vacuoles, thus providing a mechanism by which the virus may escape immune surveillance. Monocytes/macrophages are activated by Marburg virus infection as indicated by TNF-α . TNF-α is a major immune response-modifying cytokineproduced primarily by activated macrophages. Like IL-1, TNF-α induces the expression of other autocrine growth factors, increases cellular responsiveness to growth factors and induces signaling pathways that lead to proliferation. TNF-α acts synergistically with EGF and PDGF on some cell types. Like other growth factors, TNF-α induces expression of a number of nuclear proto-oncogenes as well as of several interleukins. Supematants of monocyte/macrophage cultures infected with Marburg virus increase the permeability of cultured human endothelial cell monolayers. The increase in endothelial permeability correlates with the time course of TNF-α release and can be inhibited by a TNF-α specific monoclonal antibody. Furthermore, recombinant TNF-α added at concentrations present in supematants of virus- infected macrophage cultures increases endothelial permeability indicating that TNF-α plays a critical role in mediating increased permeability, which was identified as a paraendothelial route shown by formation of interendothelial gaps. Surprisingly, the combination of viral replication in endothelial cells and monocytes/macrophages and the permeability-increasing effect of virus-induced cytokine release provide the first experimental data for a novel concept in the pathogenesis of viral hemorrhagic fever.

Further aspects of the present invention are related to the use of the MEK inhibitors for regulating and/or inhibiting virally induced TNF-α production and the use of said MEK inhibitors for the treatment of virally induced TNF-α mediated diseases. Preferably, the TNF-α production is induced by a filovirus, or a arenavirus, or a flavivirus, or a bunyavirus. Most preferably, the TNF-α production is induced by a filovirus. Thereby, the TNFe mediated diseases comprise preferably hemorrhagic fever diseases and hemorrhagic shock syndroms. The inhibitors of the protein kinases MEK are administered to a subject in need in a dosage corresponding to an effective concentration in the range of 100 nM to 10 pM, preferably in a range of 100 nM to 1 μM.

Furthermore, the present invention is directed to a method for treating or preventing virally induced TNF-α mediated diseases, especially, these virally induced diseases are associated with hemorrhagic fever and/or hemorrhagic shock syndromes. Another aspect of the present invention discloses a method for regulating and/or inhibiting of virally induced TNF-α production. Said methods comprise administering to a mammal, including a human, infected with a virus and in need of treatment, or to a mammal, including a human, at the risk of developing a virally induced disease associated with hemorrhagic fever a pharmaceutically effective amount of at least one MEK inhibitor. The mentioned virus is preferably a filovirus, arenavirus, flavivirus, or bunyavirus and most preferably a filovirus. The TNF-α mediated diseases comprise hemorrhagic fever diseases and hemorrhagic shock syndroms. Said TNF-α production may be induced by a filovirus, comprising Marburg virus, Ebola virus and Reston virus.

Especially, MEKI inhibitors are used within the disclosed methods of the present invention in order to prevent and/or treat virally induced hemorrhagic fevers and/or hemorrhagic shock syndroms, for regulating and/or inhibiting virally induced TNF-α production, and for the treatment of virally induced TNF-α mediated diseases. Preferably, the virus that induces TNF-α production, TNF-α mediated diseases, and/or hemorrhagic fever or hemorrhagic shock is a filovirus.

In 1990, mammalian p44 MAPK was cloned and referred to as extracellular signal- regulated kinase (ERK1). Since the initial discovery of ERK1, 12 MAPK genes encompassing five subfamilies have been identified in mammalian cells that are defined by sequence homology and functional similarity (cf. Table 1). The MAPK family members include ERK1/2, p38alpha, beta, gamma and delta, JNK1, 2, 3, ERK3, 4 and 5.

TABLE 1

| Acronym | Name |
|---|---|
| MKKKKs | |
| and MKKKs | |
| Rafl, A- and B-raf | |
| MEKK1, 2, 3 and 4 | MAPK/ERK kinase kinase 1-4 |
| MAPKKK5/ASK-1 | MAP Kinase kinase5/apoptosis-signal regulating |
| | kinase-1 |
| MLK1, 2 and3 | Mixed lineage kinase 1-3 |
| MKKs | |
| MEK1 and 2 | MAPK/ERK kinase |
| JNKK | JNK kinase (also known as MKK4 or SEK-1) |
| MKK3, 5, 6, 7 | MAPK kinase |
| MAPKS | |
| ERK1, 2, 3, 4 and 5 | Extracellular-signal regulated kinase |
| JNK1, 2, and 3 | c-jun N-terminal kinase |

There are seven different MKKs that regulate the MAPKs with considerable 5 specificity. MAPK/ERK (MEK) 1 and 2 regulate ERK1/2, whereas JNK kinase (JNKK; also known as SEK-1 or MKK4) and MAPK/ERK kinase 7 (MKK7) regulate JNK activity. MKK3 and MKK6 specifically phosphorylate and regulate p38 activity. MKK5 phosphorylates and regulates ERK5. There are currently 10 different groups of kinases encompassing over 22 different genes that act upstream and regulate the 10 MKKs. One family, the MAPK/ERK kinase kinases (MEKKs), directly phosphorylate and activate specific MKKs and so are valid MKKKs. Table 1 gives an overview of MAP kinases and their acronyms.

Tumor necrosis factor alpha has multiple biological functions including the prolonged activation of the collagenase and cJun genes, which are regulated via their AP-1 binding sites. Incubating human fibroblasts with TNF-α induces prolonged activation of JNK, the c-Jun kinase, which phosphorylates the transactivation domain of c-Jun. Furthermore, an immune complex kinase assay specifically demonstrates that TNF-α stimulates the activity of JNK1. TNF-α also produces a small and transient increase in extracellular signal-regulated kinase (ERK) activity, but no measured increase in Raf-1 kinase activity.

The activation of JNK by TNF-α does not correlate with Raf-1 or ERK activity. The kinetics of Raf-1, ERK, and JNK induction by epidermal growth factor, phorbol 12- myristate 13-acetate, or TNF-α indicate distinct mechanisms of activation in human fibroblasts. Tumor necrosis factor alpha activates the SAPKs (also known as Jun nuclear kinases or JNKs), resulting in the stimulation of AP-1-dependent gene transcription and induces the translocation of NF-kappa B to the nucleus. This results in the stimulation of NF-kappa B-dependent gene transcription. A potential second messenger for these signaling pathways is ceramide, which is generated when TNF-α activates sphingomyelinases.

If TNF-α remains in the body for a long time, it loses its anti tumor activity. This can occur due to polymerization of the cytokine, shedding of TNF receptors by tumor cells, excessive production of anti-TNF antibodies, found in patients with carcinomas or chronic infection, and disruptions in the alpha-2 makroglobulin proteinase system which may deregulate cytokines. Prolonged overproduction of TNF-α also results in a condition known as cachexia, which is characterized by anorexia, net catabolism, weight loss and anemia and which occurs in illnesses such as cancer and AIDS. Surprisingly it was found that filoviruses induce TNF-α overproduction which may led to viral hemorrhagic fever and/or hemorrhagic shock syndromes.

In cells that contain TNF receptors, activation of these receptors lead to turning on of many pathways that lead to toxicity in the target cell, and which culminate in apoptosis (regulated self-destruction of the cell). Multiple organ failure is more likely caused by TNF-α induced toxicity than by any other single cause. Neutral sphingomyelinase has been shown to be activated by the TNF receptor, and this, in turn, activates ceramide-activated protein kinase, which then activates the MEKIMAP kinase pathway in the target cells, probably adding to the overall toxic effects of TNF. It is known that the MEK/MAP kinase pathway is important in septic shock, and is involved at several vital points in the progression of septic shock. As disclosed in the present invention it was surprisingly found that the virally induced TNF-α overproduction optionally associated with VHF or hemorrhagic shock syndroms can be regulated using MEK inhibitors, preferably MEKI inhibitors.

Thus, the present invention is based on the mechanism of action of MEK inhibitors, most preferably MEK1 inhibitors, on the virally induced production, especially overproduction, of TNF-α which may cause fatal diseases like VHF. Most preferably, the TNF-α overproduction is induced by a Marburg virus, Ebola virus, or Reston virus.

Pharmaceutical Compositions

Yet, according to another aspect, the present invention refers to the use of at least of one of the compounds 2′-amino-3′-methoxyflavone, 2-(2-chloro-4- iodophenylamino)-N-cyclopropylmethoxy-3,4-difluorobenzamide, 1,4-diamino-2,3-dicyano-1,4-bis(2-aminophenylthio) butadiene, and 4(4-fluorophenylI2-(4-hydroxyphenyl)-5(4-pyridyl)1H-imidazole for the preparation of a pharmaceutical composition for the prophylaxis and/or treatment of inflammatory conditions.

Moreover, the present invention relates to the use of at least of one of the compounds 2′-amino-3′-methoxyflavone, 2-2-chloro-4-iodophenylamino)-N-cyclopropylmethoxy-3,4-difluorobenzamide, 1,4-diamino-2,3-dicyano-1,4-bis(2-aminophenylthio) butadiene, and 4-(4-fluorophenyl)2-4-hydroxyphenyl)-5-(4-pyridyly1H-imidazole for the preparation of a pharmaceutical composition for the prophylaxis and/or treatment of virally induced hemorrhagic fever and/or hemorrhagic shock syndromes.

Another aspect of the present invention relates to a pharmaceutical composition comprising at least one of the compounds 2′-amino-3′-methoxyflavone, 2-(2-chloro-4-iodophenylamino)-N-cyclopropylmethoxy-3,4-difluorobenzamide, 1,4-diamino-2,3-dicyano-1,4-bis(2-aminophenylthio)butadiene, and 4-(4-fluorophenyl)-2-4-hydroxyphenylI5-(4-pyridyl)-1H-imidazole as an active ingredient, optionally together with one or more pharmaceutically acceptable carriers, excipients, adjuvents, and/or diluents.

A still further aspect of the present invention relates to pharmaceutical compositions comprising at least one MEK inhibitor, most preferably MEK1 inhibitor, as an active ingredient and, optionally, at least one pharmaceutically acceptable carrier, excipient, adjuvent and/or diluent.

The MEK inhibitors can also be administered in form of their pharmaceutically active salts optionally using substantially nontoxic pharmaceutically acceptable carrier, excipients, adjuvents or diluents. The medications of the present invention are prepared in a conventional solid or liquid carrier or diluent and a conventional pharmaceutically-made adjuvant at suitable dosage level in a known way. The preferred preparations are in administratable form which is suitable for oral application. These administratable forms, for example, include pills, tablets, film tablets, coated tablets, capsules, powders and deposits. Other than oral adminisratable forms are also possible. The MEK inhibitors or pharmaceutical preparations containing said MEK inhibitors may be administered by any appropriate means, including but not limited to injection (intravenous, intraperitoneal, intramuscular, subcutaneous) by absorption through epithelial or mucocutaneous linings (oral mucosa, rectal and vaginal epithelial linings, nasopharyngial mucosa, intestinal mucosa); orally, rectally, transdermally, topically, intradermally, intragastrally, intracutanly, intravaginally, intravasally, intranasally, intrabuccally, percutanly, sublingually, or any other means available within the pharmaceutical arts.

Within the disclosed methods the pharmaceutical compositions of the present invention, containing at least one MEK inhibitors, preferably MEK1 inhibitor, or pharmaceutically acceptable salts thereof as an active ingredient will typically be administered in admixture with suitable carrier materials suitably selected with respect to the intended form of administration, i.e. oral tablets, capsules (either solid-filled, semi-solid filled or liquid filled), powders for constitution, oral gels, elixirs, dispersible granules, syrups, suspensions, and the like, and consistent with conventional pharmaceutical practices. For example, for oral administration in the form of tablets or capsules, the active drug component may be combined with any oral nontoxic pharmaceutically acceptable inert carrier, such as lactose, starch, sucrose, cellulose, magnesium stearate, dicalcium phosphate, calcium sulfate, talc, mannitol, ethyl alcohol (liquid forms) and the like. Moreover, when desired or needed, suitable binders, lubricants, disintegrating agents and coloring agents may also be incorporated in the mixture. Powders and tablets may be comprised of from about 5 to about 95 percent inventive composition.

Suitable binders include starch, gelatin, natural sugars, corn sweeteners, natural and synthetic gums such as acacia, sodium alginate, carboxymethyl-cellulose, polyethylene glycol and waxes. Among the lubricants there may be mentioned for use in these dosage forms, boric acid, sodium benzoate, sodium acetate, sodium chloride, and the like. Disintegrants include starch, methylcellulose, guar gum and the like. Sweetening and flavoring agents and preservatives may also be included where appropriate. Some of the terms noted above, namely disintegrants, diluents, lubricants, binders and the like, are discussed in more detail below.

Additionally, the compositions of the present invention may be formulated in sustained release form to provide the rate controlled release of any one or more of the components or active ingredients to optimize the therapeutic effects, i.e. antihistaminic activity and the like. Suitable dosage forms for sustained release include layered tablets containing layers of varying disintegration rates or controlled release polymeric matrices impregnated with the active components and shaped in tablet form or capsules containing such impregnated or encapsulated porous polymeric matrices.

Liquid form preparations include solutions, suspensions and emulsions. As an example may be mentioned water or water-propylene glycol solutions for parenteral injections or addition of sweeteners and opacifiers for oral solutions, suspensions and emulsions. Liquid form preparations may also include solutions for intranasal administration.

Aerosol preparations suitable for inhalation may include solutions and solids in powder form, which may be in combination with a pharmaceutically acceptable carrier such as inert compressed gas, e.g. nitrogen.

For preparing suppositories, a low melting wax such as a mixture of fatty acid glycerides such as cocoa butter is first melted, and the active ingredient is dispersed homogeneously therein by stirring or similar mixing. The molten homogeneous mixture is then poured into convenient sized molds, allowed to cool and thereby solidify.

Also included are solid form preparations which are intended to be converted, shortly before use, to liquid form preparations for either oral or parenteral administration. Such liquid forms include solutions, suspensions and emulsions.

The MEK inhibitors of the present invention may also be deliverable transdermally. The transdermal compositions may take the form of creams, lotions, aerosols and/or emulsions and can be included in a transdermal patch of the matrix or reservoir type as a re conventional in the art for this purpose.

The term capsule refers to a special container or enclosure made of methyl cellulose, polyvinyl alcohols, or denatured gelatins or starch for holding or containing compositions comprising the active ingredients. Hard shell capsules are typically made of blends of relatively high gel strength bone and pork skin gelatins. The capsule itself may contain small amounts of dyes, opaquing agents, plasticizers and preservatives.

Tablet means compressed or molded solid dosage form containing the active ingredients with suitable diluents. The tablet can be prepared by compression of mixtures or granulations obtained by wet granulation, dry granulation or by compaction well known to a person skilled in the art.

Oral gels refers to the active ingredients dispersed or solubilized in a hydrophillic semi-solid matrix.

Powders for constitution refers to powder blends containing the active ingredients and suitable diluents which can be suspended in water or juices.

Suitable diluents are substances that usually make up the major portion of the composition or dosage form. Suitable diluents include sugars such as lactose, sucrose, mannitol and sorbitol, starches derived from wheat, corn rice and potato, and celluloses such as microcrystalline cellulose. The amount of diluent in the composition can range from about 5 to about 95% by weight of the total composition, preferably from about 25 to about 75%, more preferably from about 30 to about 60% by weight.

The term disintegrants refers to materials added to the composition to help it break apart (disintegrate) and release the medicaments. Suitable disintegrants include starches, “cold water soluble” modified starches such as sodium carboxymethyl starch, natural and synthetic gums such as locust bean, karaya, guar, tragacanth and agar, cellulose derivatives such as methylcellulose and sodium carboxymethylcellulose, microcrystalline celluloses and cross-linked microcrystalline celluloses such as sodium croscarmellose, alginates such as alginic acid and sodium alginate, clays such as bentonites, and effervescent mixtures. The amount of disintegrant in the composition can range from about 2 to about 20% by weight of the composition, more preferably from about 5 to about 10% by weight.

Binders characterize substances that bind or “glue” powders together and make them cohesive by forming granules, thus serving as the “adhesive” in the formulation. Binders add cohesive strength already available in the diluent or bulking agent. Suitable binders include sugars such as sucrose, starches derived from wheat, corn rice and potato; natural gums such as acacia, gelatin and tragacanth; derivatives of seaweed such as alginic acid, sodium alginate and ammonium calcium alginate; cellulosic materials such as methylcellulose and sodium carboxymethylcellulose and hydroxypropylmethylcellulose; polyvinylpyrrolidone; and inorganics such as magnesium aluminum silicate. The amount of binder in the composition can range from about 2 to about 20% by weight of the composition, more preferably from about 3 to about 10% by weight, even more preferably from about 3 to about 6% by weight.

Lubricant refers to a substance added to the dosage form to enable the tablet, granules, etc. after It has been compressed, to release from the mold or die by reducing friction or wear. Suitable lubricants include metallic stearates such as magnesium stearate, calcium stearate or potassium stearate; stearic acid; high melting point waxes; and water soluble lubricants such as sodium chloride, sodium benzoate, sodium acetate, sodium oleate, polyethylene glycols and d′l-leucine. Lubricants are usually added at the very last step before compression, since they must be present on the surfaces of the granules and in between them and the parts of the tablet press. The amount of lubricant in the composition can range from about 0.2 to about 5% by weight of the composition, preferably from about 0.5 to about 2%, more preferably from about 0.3 to about 1.5% by weight.

Glidents are materials that prevent caking and improve the flow characteristics of granulations, so that flow is smooth and uniform. Suitable glidents include silicon dioxide and talc. The amount of glident in the composition can range from about 0.1% to about 5% by weight of the total composition, preferably from about 0.5 to about 2% by weight.

Coloring agents are excipients that provide coloration to the composition or the dosage form. Such excipients can include food grade dyes and food grade dyes adsorbed onto a suitable adsorbent such as clay or aluminum oxide. The amount of the coloring agent can vary from about 0.1 to about 5% by weight of the composition, preferably from about 0.1 to about 1%.

Techniques for the formula and administration of the MEK inhibitors of the present invention may be found in “Remington's Pharmaceutical Sciences” Mack Publishing Co., Easton PA. A suitable composition comprising at least one MEK inhibitor, especially MEK1 inhibitor, of the invention may be a solution of the compound in a suitable liquid pharmaceutical carrier or any other formulation such as tablets, pills, film tablets, coated tablets, dragees, capsules, powders and deposits, gels, syrups, slurries, suspensions, emulsions, and the like.

A therapeutically effective dosage of a MEK inhibitors refers to that amount of the compound that results in an at least partial inhibition of viral mediated TNF-α release. Toxicity and therapeutic efficacy of such compounds can be determined by standard pharmaceutical, pharmacological, and toxicological procedures in cell cultures or experimental animals for determining the LD50 (the dose lethal to 50% of the population) and the ED50 (the dose therapeutically effective in 50% of the population). The dose ratio between toxic and therapeutic effect is the therapeutic index and can be expressed as the ratio between LD50 and ED50. The dosage of the compound lies preferably within a range of circulating concentrations that include the ED50 with little or no toxicity. More preferably, the dosage of the compound corresponds to an effective concentration in the range of 0.1-5 μM. The actual amount of the composition administered will be dependent on the subject being treated, on the subject's weight, the severity of the affliction, the manner of administration and the judgment of the prescribing physician.

### DESCRIPTION OF THE FIGURES

FIGS. 1a to 1d show the effect of two MEK inhibitors at different concentrations on the TNF-α release of Ebola (EBO) or Marburg (MBG) virus infected peripheral blood mononuclear cells (PBMC) of two different donors. As a control, the above-mentioned PBMC's were incubated with the supematants of non virus-producing Vero E6 cells (MOCK). 21 hours post infection (pi), mock-infected PBMC's did not release TNF-α in significant amounts to the supematants irrespective of absence or presence of the inhibitor (Figs. la to Id, MOCK columns 1 to 5). Infection of PBMC's with Marburg or Ebola virus in the absence of any inhibitor (positive control) resulted in a dramatic release of TNF-α to the supermatant 21 h post infection (FIGS. 1a to 1d, MBG column 1; FIGS. 1a to 1d, EBO column 1). In the presence of the respective inhibitor TNF-α release to the supermatant was dramatically inhibited in a dose-dependant manner at between 100 nM to 1 μM concentrations (FIGS. 1a to 1d, MBG columns 2 to 5; FIGS. 1a to 1d, EBO columns 2 to 5). The effect was observed for both donors. These results suggest, that both compounds can efficiently inhibit TNF-α alpha release induced by Ebola and Marburg viruses.

FIGS. 2a to 2c demonstrate the effect of the MEK-inhibitor 1,4-diamino-2,3-dicyano-1,4-bis (2-aminophenylthio)butadiene (compound 3) on replication of Ebola and Marburg virus in a plaque test in VeroE6 cells (FIGS. 2a and 2b) and human macrophages (FIG. 2c). Presence or absence of compound 3 does not have an obvious effect on filovirus replication in the above mentioned cells reflected by the presence of similar amounts of plaques 5 days post infection.

FIG. 3 shows the inhibitory effect of the MEK-inhibitor 1,4-diamino-2,3-dicyano-1,4-bis(2-aminophenylthio) butadiene (compound 3) on the release of IL-Ibeta, a cytokine commonly upregulated in inflammatory processes. IL-I beta is not detected in mock-infected VeroE6 cells despite presence (FIG. 3 Mock + column) or absence (FIG. 3 MOCK - column) of the MEK-inhibitor compound 3. While infection of VeroE6 cells with Marburg or Ebola virus results in a prominent increase of IL-1 beta 22 h post infection in the supermatant of the cells (FIG. 3 MBG_{“}-” column; FIG. 3 EBO-Z _{“}-” column), presence of the MEK-inhibitor compound 3 dramatically reduces the release of IL-1 beta to the supermatant (FIG. 3 MBG ,,+column; FIG. 3 EBO _{“}+” column).

FIGS. 4a to 4c show the dose-dependent inhibitory effect of compounds 1 and 3 (FIG. 4a) and the inhibitory effect of an additional MEK-inhibitor (2-(2-chloro4-iodophenylamino)-N-cyclopropylmethoxy-3,4-difluoro-benzamide, compound 2; FIGS. 4b and 4c) at different concentrations on the TNF-α alpha release of Ebola (EBO) or Marburg (MBG) virus infected peripheral blood mononuclear cells (PBMC) of three different donors 20 hours post infection.

FIGS. 5a to 5d demonstrate the inhibitory effects of 2′-amino-3′-methoxyflavone (compound 1), 2-(2-chloro-4-iodophenylamino)-N-cyclopropylmethoxy-3,4-difluorobenzamide (compound 2), and 1 ,4-diamino-2,3 dicyano-1 ,4-bis(2-aminophenylthio)buta-diene (compound 3) on the virally (MBG Marburg virus; EBO Ebola virus) induced TNF-α alpha release in primary human macrophages from two donors, 17 hours after viral induction. In sharp contrast, the compounds 1, 2 and 3 did not reduce bacterial LPS-induced TNF-α alpha release (see columns 5 in

FIGS. 5a and 5b) in comparison to viral induced TNF-α release (see columns 2 and 3 in FIGS. 5a and 5b). Compound 4, the p38 kinase inhibitor 4i4-fluorophenyl)-2-(4hydroxyphenyl)-5-(4pyridyly1 H-imidazole did not significantly inhibit Marburg oder Ebola virus induced TNF-α alpha release at usually effective concentrations (FIG. 5a last five columns numbers 2 and 3). FIGS. 5c and 5d represent magnifications of the viral data from FIGS. 5a and 5b without the data for the bacterial LPS-induced TNF-α alpha release.

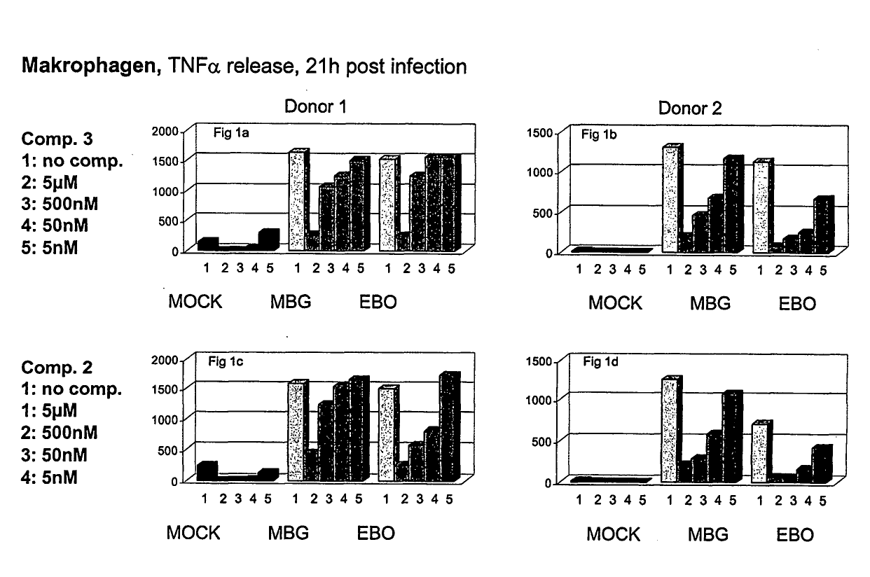

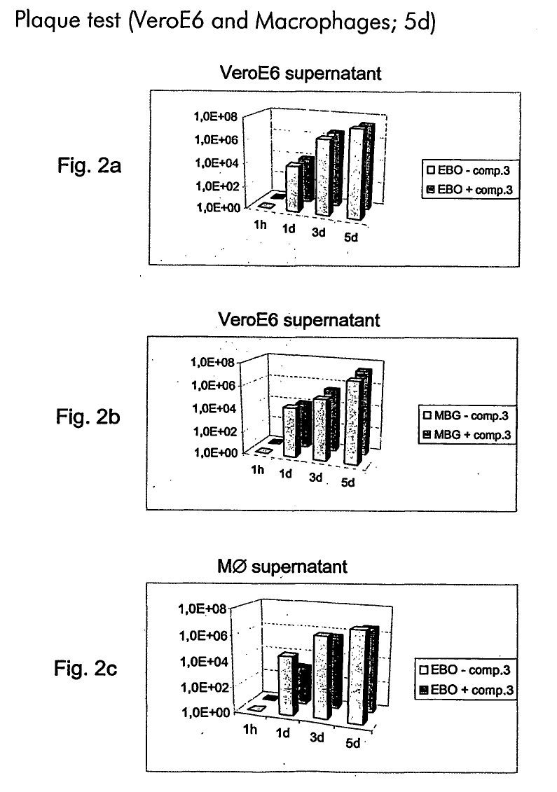

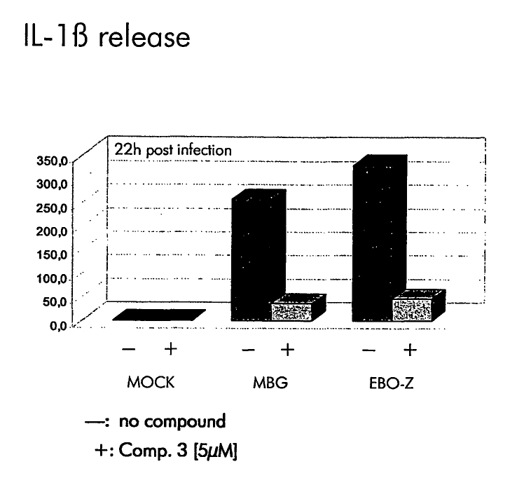

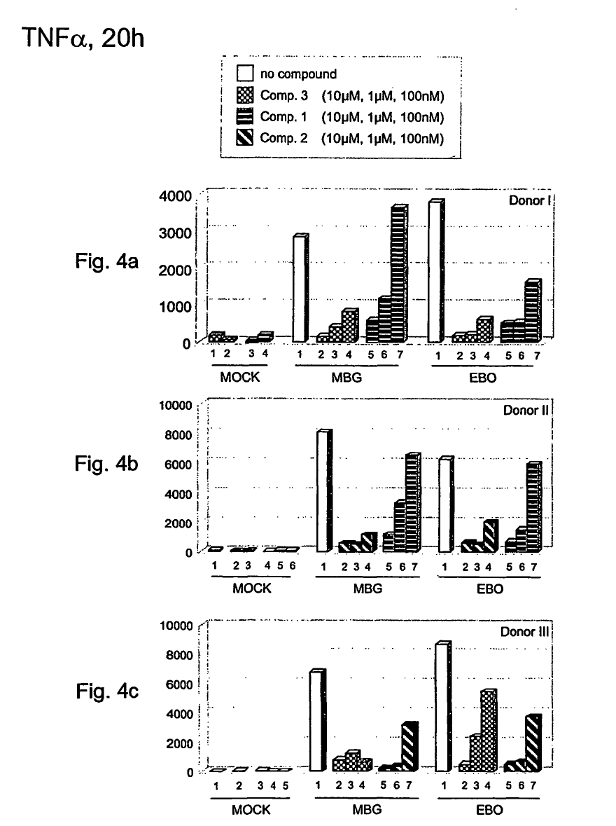

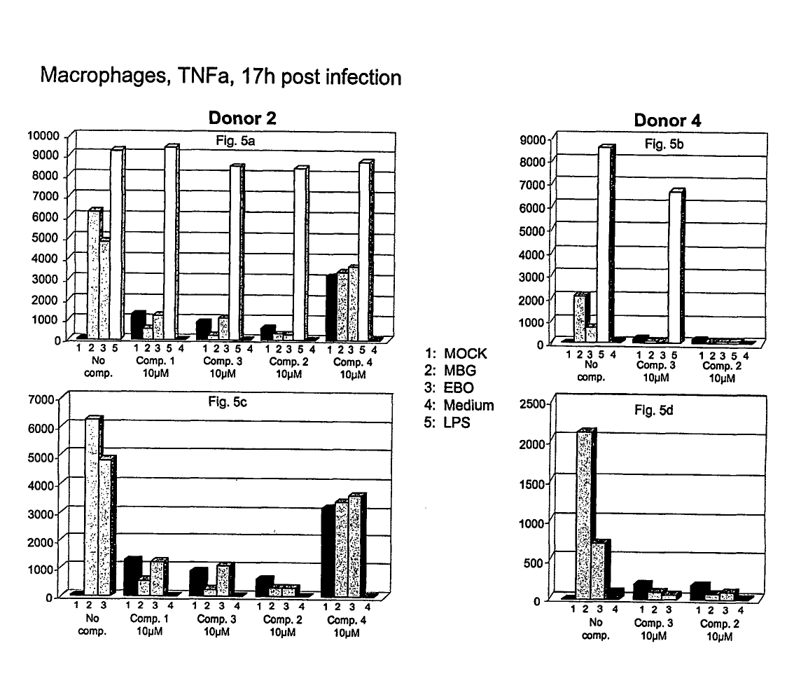

The reaction conditions of the tests shown in the figures were as follows. The cells were incubated with the inhibitor 30 minutes before infection and stimulation (with lipopolysaccaride, LPS), respectively. The temperature was 37° C. in each case.

In the Figures, the units given in the graphs refer to picogram TNF-α per ml and IL-beta, respectively, in the supernatant with cells.

The “medium” designated in the Figures is a RPMI 1640 medium supplemented with 2 mM glutamine, 100 U/ml penicillin, 100 pjglml streptomycin, 2 mM non-essential amino acids, 2 mM pyruvate, and 5% human AB serum. With the term “Mock”, supematants of VeroE cells not infected with MBGV and EBOV cells are denoted. The Mock tests were run under the same conditions as the tests with the MBGV and EBOV cells, i.e. in the same medium (DMEM, 2% FCS) and for the same incubation period.

“LPS” means lipopolysaccaride from E. coli and obtained from Sigma, Deisenhofen, Germany. LPS was used as a positive control for TNFα expression based on bacterial stimulation.

### Examples

Materials and Methods

Primary human peripheral blood mononuclear cells were prepared from buffy coats of healthy donors. The mononuclear cell fraction was separated and finally resuspended in RPMI 1640 medium supplemented with 5% heat-inactivated pooled human serum. For separation of adherent monocytes/macrophages from nonadherent cells, 5 × 106 cells were plated into 24-well tissue culture plates, incubated for 1 h at 37° C., and subsequently washed to remove nonadherent cells. Adherent cells were cultured for 7 days in RPMI 1640 medium containing 5% human serum in order to obtain a macrophage-like morphology. Monocytes/macrophages were infected with virus strains at an MOI of 10 after 7 days in cell culture. Adsorption of viral particles was performed for 1 h at 37° C. Subsequently, the inoculum was removed, and the cells were washed twice with phosphate-buffered saline (PBS). RPMI 1640 medium supplemented with 5% human serum from healthy donors and the respective concentrations of various chemical inhibitors was added, and incubation proceeded for 12, 17, or 24h, respectively, at 37° C. After incubation times, tissue culture supernatants were collected and determination of TNF-α in the culture supematants was performed with an enzyme-linked immunosorbent assay (ELISA) according to the manufacturer's protocol.

Viruses and cell lines: In this study we used the Musoke strain of MBGV, the Mayinga strain of the Zaire species of EBOV (EBOV-Zaire), and the Z-strain of Sendai virus. MBGV and EBOV virus stocks were kindly provided by the Special Pathogens Branch, Centers for Disease Control and Prevention, Atlanta, USA. MBGV and EBOV stocks were freshly prepared in Vero E6 cells (ATCC 1568). Mock-infected Vero E6 cells were treated the same way in order to prepare a control (mock stock). Vero E6 cells were cultured in Dulbecco's Minimum Essential Medium (DMEM) (GIBCO-BRL, Germany) supplemented with 10% fetal calf serum

(Biochrom, Germany), penicillin (100 U/ml), streptomycin (100 μg/mI) and L-glutamine (2 mM). For virus propagation DMEM with 2% fetal calf serum was used. Sendai virus were amplified in the allantoic cavity of 11-day-old embryonated chicken eggs.

Endotoxin test Prior to use, all virus stocks and media were analyzed for endotoxin presence using the ‘Limulus amebocyte lysate test’ (QCL-1000; BioWhittaker, Walkersville, MD. U.S.A.). All compounds and media used in this study contained less than 0.3 EU/ml which was less or equivalent to the amounts found in the mock stock used as controls for the experiments.

Isolation of peripheral blood mononuclear cells (PBMC): Human PBMCs were obtained from leukocyte-rich buffy coats of healthy donors (Blutbank, Marburg). Cells in fresh, single buffy coats were fractionated by centrifugation on Ficoll-Paque gradient (Pharmacia), and blood mononuclear cells (2 × 107 cells/well) were allowed to adhere onto 24-well plates (Primaria, Falcon) for 1h in RPMI 1640 medium supplemented with glutamine (2 mM), penicillin (100 U/ml), streptomycin (100 μg/ml), nonessential amino acids (2 mM), pyruvate (2 mM), and 5% human AB-serum. Plastic-adhered monocytes were washed with PBS and differentiated into macrophages by culturing them for 7 days at 37° C. in a humidified (95%) 5% CO₂ air atmosphere.

Inhibitors: The inhibitors 2′-amino-3′-methoxyflavone (Compound 1), 2-2-chloro-4-iodophenylamino)-3,4-difluorobenzamide (Compound 2), 1,4-diamino-2,3-dicyano-1,4-bis(2-aminophenylthio)butadiene (Compound 3), and 4-(4-fluorophenylI2-(4hydroxyphenyl)-5-(4-pyridyl)-1 H-imidazole (Compound 4) have been obtained from Calbiochem and were dissolved in dimethyl sulphoxide.

Assaying of cytotoxicity. The cytotoxic effect of the inhibitors was measured by the MTT-assay as described elsewhere. Twenty four, forty eight, and seventy two hours after the final treatment, 100 μl of MTT reagent (Sigma) was added to each sample for 4 h, then 100 μl of solubilization solution (20% SDS/50% DMF) was added to the cells for 14h. Plates were analyzed on a microplate reader at 595 nm.

Infection and treatment of macrophages: The infection was performed on day 7 post seeding at a MOI of 10. Where indicated cells were incubated with the appropriate inhibitor 30 min prior to infection. After an adsorption period of 1 hour, the inoculum was replaced by new medium (RPMI containing 5% human AB-serum) and the cultures were incubated for various times at 37° C. in a incubator (humidity 95%). Where indicated, cells were treated with 2′-amino-3′-methoxyflavone, 2-(2-chloro-4-iodophenylamino)N-cyclopropylmethoxy-3,4-difluorobenzamide, 1,4-diamino-2,3-dicyano-1,4bis(2-aminophenylthio)butadiene, 4-(4fluorophenyl)-2-(4-hydroxyphenyl)5-(4-pyridyly1 H-imidazole, respectively, 45min prior to infection or LPS-stimulation. The inhibitors were also present during infection and subsequent incubation periods.

Detection of Cytokines: The supematants from the infected or mock-infected cell cultures were removed, clarified from cell debris by centrifugation (8000 × g, 4° C., 10 minutes) and stored at −80° C. All samples were tested in duplicates for the presence of cytokines after thawing them only once using commercial ELISA systems (human IL-6, TNF-α ELISA Kit: Promocell, human gro-α, IL-1 β ELISA: R&D Systems). Since the samples were not inactivated, the analyses were conducted in a biosafety level 4 (BSL4) containment laboratory.

Westem blot analysis

To detect ERK phosphorylation by Western blofting infected or stimulated macrophages were washed with cold PBS and harvested at the indicated times by lysis in cold lysis buffer (10 mM Tris, pH 7.2, 150 mM NaCI, 1% TritonX-100, 1% sodium deoxycholate, 1 mM PMSF, 50 mM sodium fluoride, I mM sodium orthovanadate, 50 μg/ml aprotinin, and 50 μg/mI leupeptin). After centrifugation supematants were assayed for protein by the Amido-Schwarz method as described by Schnittler et al., Replication of Marburg virus in human endothelial cells: a possible mechanism for the development of viral hemorrhagic disease. J Clin Invest 1993;91:1301-1309. Protein samples (1 μg) were analyzed by SDS-polyacrylamide gel electrophoresis on 10% Tris gels. Protein was electrotransferred to Polyvinylidene difluoride membrane (ImmobilonP membrane, Millipore, ). Membranes were rinsed in 10 mM Tris-CI, pH 7.5, 100 mM NaCI, 0,1% Tween-20 (TBS-T), incubated in blocking buffer (TBS-T with 3% BSA) for I h and probed with a polyclonal phosphospecific antibody against ERK1 and ERK2 (New England Biolabs) over night at 4° C. Detection of bands was carried out according to the manufacturer's protocol.

Where indicated in the legends to the figures, blots were stripped by incubation at 370° C. in Restore Western Blot Stripping Buffer (Pierce) for 30 min, followed by washing in TBS-T and blocking before reprobing with an antibody against ERK1 and ERK2 (New England Biolabs).

Plaque Assa: Confluent Vero E6 cell monolayers cultured in six-well plates were infected with clarified tissue culture supermatant of MBGV- or EBOV- infected Vero E6 cells and macrophages, respectively, at 10-fold dilutions ranging from 103 to 10⁻⁸. After 1 hour cells were washed and covered with an overlay of DMEMW1.8% LMP Agarose/2% FCS. Plaques were stained up to 5 days post infection with 0.1% crystal violet in a 10% formaldehyde solution.

Results

The MEKI inhibitors 2′-amino-3′-methoxyflavone, 2-(2-chloro4-iodophenylamino)-N-cyclopropylmethoxy-3,4-difluorobenzamide, and 1,4-diamino-2,3-dicyano-1,4-bis(2-aminophenylthio(butadiene respectively, inhibited very strongly and dose-dependently the Marburg virus or Ebola virus induced TNFα production at least between 100 nM and I pM concentrations.

The MEKI inhibitors 2′-amino-3′-methoxyflavone, 2-(2-chloro-4-iodophenylamino)-N-cyclopropylmethoxy-3,4-difluorobenzamide, and 1,4-diamino-2,3-dicyano-1,4-bis(2-aminophenylthio)butadiene, or the p38 kinase inhibitor 4-(4-fluorophenyl{2-(4-hydroxyphenyl)-5-(4-pyridyly1 H-imidazole had no inhibitory effect at all on the bacterial LPS-induced TNF-α production at 10 μM concentrations.

The p38 kinase inhibitor 4-(4-fluorophenyl2-(4-hydroxyphenyl)5-(4-pyridyly1 H-imidazole did not significantly inhibit the Marburg virus or Ebola virus induced TNF-α production at usually effective concentrations, indicating that the tested MEK1 inhibitors are powerful and selective inhibitors of filovirus-induced TNF-α production.

1. Use of at least one compound selected from the group consisting of 2′-amino-3′-methoxyflavone, 2-(2-chloro-4-iodophenylamino)-N-cyclopropylmethoxy-3,4-difluorobenzamide, 1,4-diamino-2,3-dicyano-1,4-bis (2-aminophenylthio)butadiene, and 4-4-fluorophenyl)-2-(4-hydroxyphenyl)-5-(4-pyridyl)- H-imidazole for the prophylaxis and/or treatment of inflammatory conditions.

2. Use of at least one compound selected from the group consisting of 2′-amino-3′-methoxyflavone, 242-chloro4iodophenylamino)-N-cyclopropylmethoxy-3,4-difluorobenzamide, 1,4-diamino-2,3-dicyano-1,4-bis (2-aminophenylthio) butadiene, and 4-(4-fluorophenyl)-2(4-hydroxyphenyl)-5-(4-pyridyly H-imidazole for prophylaxis and/or treatment of virally induced hemorrhagic fever and/or hemorrhagic shock syndromes.

3. Use of at least one MEK inhibitor for prophylaxis and/or treatment of virally induced hemorrhagic fever and/or hemorrhagic shock syndromes and/or inflammatory conditions.

4. Use according to one of claims 2 or 3, wherein the hemorrhagic fever or the hemorrhagic shock syndromes are induced by filoviruses.

5. Use of at least one compound selected from the group consisting of 2′-amino-3′-methoxyflavone, 242-chloro-4-iodophenylamino)-N-cyclopropylmethoxy-3,4-difluorobenzamide, 1,4-diamino-2,3-dicyano-1,4-bis (2-aminophenylthio) butadiene, and 4-(4-fluorophenyl2-(4-hydroxyphenyl5-(4-pyridyly H-imidazole for regulating and/or inhibiting virally induced TNF-α production.

6. Use of at least one MEK inhibitor for regulating and/or inhibiting virally induced TNF-α production.

7. Use of at least one MEK inhibitor for the treatment of virally induced TNF-α mediated diseases.

8. Use according to claim 7, wherein the TNF-α mediated diseases comprise hemorrhagic fever diseases and hemorrhagic shock syndromes.

9. Use according to one of claims 7 or 8, wherein the TNFα production is induced by filoviruses.

10. Use according to one of claims 4 or 9, wherein the filovirus is a Marburg virus, Ebola virus or a Reston virus.

11. Use according to one of claims 3, 4, or 6 to 10, wherein the MEK inhibitor is a MEK1 inhibitor.

12. Use according to one of claims 1 to 11, wherein the compound or MEK inhibitor is administered in a dosage corresponding to an effective concentration in the range of 100 nM to 1 μM.

13. Method for treating or preventing virally induced hemorrhagic fever and hemorrhagic shock syndromes, said method comprising administering to a mammal infected with a virus and in need of treatment, or to a mammal at the risk of developing a virally induced disease associated with hemorrhagic fever a pharmaceutically effective amount of at least one compound selected from the group consisting of 2′-amino-3′-methoxyflavone, 2-(2-chloro-4-iodophenylamino)-N-cyclopropylmethoxyflavone, 2-(2-chloro-4-iodophenylamino)-N-cyclopropylmethoxy-3,4-difluorobenzamide, 1,4-diamino-2,3-dicyano-1,4-bis(2-aminophenylthio) butadiene, and 4-(4-5 fluorophenylI2-(4-hydroxyphenyl)-5q4-pyridyl)-1H-imidazole, or at least one MEK inhibitor.

14. Method according to claim 13, wherein the hemorrhagic fever or the hemorrhagic shock syndromes are induced by filoviruses.

15. Method for regulating and/or inhibiting virally induced TNF-α production, said method comprising administering to a mammal infected with a virus and in need thereof a pharmaceutically effective amount of at least one compound selected from the group consisting of 2′-amino-3′-methoxyflavone, 2-(2-chloromiodophenylamino)-N-cyclopropylmethoxy-3,4-difluorobenzamide, 1,4-diamino-2,3dicyano-1,4-bis(2-aminophenylthio) butadiene, and 4-(4-4-fluorophenyl)2-(4-hydroxyphenyl)-5(4-pyridyl H-imidazole, or at least one MEK inhibitor.

16. Method for treating or preventing virally induced TNF-α mediated diseases, said method comprising administering to a mammal infected with a virus and in need of treatment, or to a mammal at the risk of developing a virally induced disease associated with hemorrhagic fever a pharmaceutically effective amount of at least one compound selected from the group consisting of 2′-amino-3′-methoxyflavone, 2-(2-chloro-4-iodophenylamino)-N-cyclopropylmethoxy-3,4-difluorobenzamide, 1,4-diamino-2,3-dicyano-1,4-bis(2-aminophenylthio) butadiene, and 4(4-fluorophenyl)-2-(4-hydroxyphenyl)-5-(4-pyridyly1 H-imidazole, or at least one MEK inhibitor.

17. Method according to claim 16, wherein the TNF-α mediated diseases comprise hemorrhagic fever diseases and hemorrhagic shock syndromes.

18. Method according to claim 15, wherein the TNF-α production is induced by filoviruses.

19. Method according to one of claims 14 or 18, wherein the filovirus is a Marburg virus, Ebola virus or a Reston virus.

20. Method according to one of claims 13, 15, or 16 wherein the MEK inhibitor is a MEK1 inhibitor.

21. Method according to one of claims 13 to 20, wherein the compound is administered in a dosage corresponding to an effective concentration in the range of 100 nM to 1 μM.

22. Use of at least of one of the compounds 2′-amino-3′-methoxyflavone, 2-(2-chlororiodophenylamino) N-cyclopropylmethoxy-3,4-difluorobenzamide, 1,4-diamino-2,3-dicyano-1,4-bis(2-aminophenylthio) butadiene, and 4(4-fluorophenyl)-2-(4-hydroxyphenyl)5-4-pyridyly1 H-imidazole for the preparation of a pharmaceutical composition for the prophylaxis and/or treatment of inflammatory conditions.

23. Use of at least of one of the compounds 2′-amino-3′-methoxyflavone, 2-(2-chloro-4-iodophenylaminoYN-cyclopropylmethoxy-3,4-difluorobenzamide, 1,4-diamino-2,3-dicyano-1,4-bis(2-aminophenylthio) butadiene, and 4<4-fluorophenyl)-2-(4-hydroxyphenyl)-5(4-pyridyl)-1 H-imidazole for the preparation of a pharmaceutical composition for the prophylaxis and/or treatment of virally induced hemorrhagic fever and/or hemorrhagic shock syndromes.

24. Pharmaceutical composition comprising at least one of the compounds 2′-amino-3′-methoxyflavone, 242-chloro~iodophenylamino)-N-cyclopropylmethoxy-3,4-difluorobenzamide, 1,4-diamino-2,3-dicyano-1,4-bis(2-aminophenylthio) butadiene, and 4-(4-fluorophenyl)-2-(4-hydroxyphenyl)5-4pyridyl)I H-imidazole as an active ingredient, optionally together with one or more pharmaceutically acceptable carriers, excipients, adjuvents, and/or diluents.

25. Pharmaceutical composition comprising at least one MEK inhibitor as an active ingredient, optionally together with one or more pharmaceutically acceptable carriers, excipients, adjuvents, and/or diluents.
